(19) United States
(12) Patent Application Publication
(10) Pub. No.: US 2005/0260138 A1
(43) Pub. Date: Nov. 24, 2005

(54) Producton and use of a gaseous vapor disinfectant

(76) Inventors: Virgil Flanigan, Rolla, MO (US); Shubhender Kapila, Rolla, MO (US); Robert I. Bolla, Youngstown, OH (US)

Correspondence Address: LATHROP & GAGE LC, 4845 PEARL EAST CIRCLE, SUITE 300, BOULDER, CO, 80301, US

(21) Appl. No.: 10/851,780

(22) Filed: May 21, 2004

(51) Int. Cl. A61K 31/22; A61L 9/04

(52) U.S. Cl. 424/45; 514/549

(57) ABSTRACT

Smoke or vapor generating system disperses an airborne biocidal oil, such as methyl soyate, to decontaminate and area or object. The airborne forms of methyl soyate are have broad spectrum efficacy against a wide variety of microscopic pathogens including viruses, bacteria and fungi.

### BACKGROUND

1. Field of the Invention

The invention pertains to the use of misting or vaporizing disinfectants, for example, over large areas, such as a room, building, industrial complex, commuter rail line, or battlefield. More specifically, the disinfectant may be a vegetable oil derivative or petroleum oil.

2. Description of the Related Art

Microbial contamination of human environments always has presented a threat to health, and now poses a potential for bioterrorism directly against humans and against their food supply chain. This is exemplified by outbreaks of polio, bubonic plague, tuberculosis, cholera, salmonellosis, and more recently legionella and hemorrhagic Escherichia coli O157:H7. Cholera and hepatitis—endemic in many areas—may be spread by fecal matter on produce. Salmonella spp. and E. coli contamination of meat and other foods is a continuing problem. In recent years the danger of microbial contamination has become more evident with the increase in food borne diseases, nosocomial infections in hospitals and the threat of bioterrorism. A recent instance of bioterrorism occurred when the United States mail stream was contaminated with weapons-grade Bacillus anthracis creating problems of cleaning not only the mail but the areas in which mail was handled and to which it was delivered.

Microbes also are significant problems to agricultural production as they are both plant and animal pathogens, and as potential contaminates of the human food chain. If not controlled, common animal symbiots can increase to a concentration in which they threatened the health of both the producer and the consumer. Salmonella contamination of poultry has long been a problem. This microorganism is a natural bacterium found associated with poultry and a natural contaminate of the production system. In recent years changes in how poultry are raised and processed for market has allowed this organism to enter the human food stream as a result of improper preparation of the food product. Considerable efforts have been put forward to manage Salmonella and decrease its threat to human health. Thus, methods have been developed to decontaminate eggs as well as carcasses during or after slaughter, for example, by irradiation.

Without an effective way to eliminate the pathogen, methods also have been developed to immunize poultry against Salmonella. These include production of non-infective isolates of Salmonella as a vehicle to induce immunity when fed to, sprayed on, or in some other way applied to young chicks, as reported in U.S. Pat. No. 5,868,998 issued to Larosse et al.

A study of Escherichia coli O157:H7 may characterize one problem of a veterinary-based microorganism that threatens human health. Antibiotics have long been used in small amounts to promote animal growth. This use has led to development of antibiotic resistant microorganisms and in cattle to the alteration of microflora in the gut. Thus there has been an increase in the prevalence of E. coli O157-H7. This organism is normal to cattle gut and is not harmful to cattle, but becomes hemorrhagic in humans (Madigan et al, 2000). Several outbreaks of E. coli O157:H7 have occurred in North America. These have arisen from meat contaminated during the processing of the carcasses. The infection for the most part has been associated with undercooked meat served in fast food restaurants. These outbreaks have led to death, particularly among children and the elderly, and have been expensive through product recall to the producer. Methods have been developed to decontaminate carcasses after slaughter or animals during the slaughter process including treatment with steam or hot water, but again the problem has not been addressed at the source by decontamination of the slaughter or processing facility because there is no effective and safe way to accomplish this.

Current methods for decontamination of large spaces, such as office buildings; hospital rooms; surgical suites; cargo vessels including trucks; ships and planes, etc. . . . ; involve cleaning surfaces with chlorine based solvents, glutaraldehyde, formaldehyde, commercial disinfectants such as Lysol® and Pinesol®, and alcohol based products or fumigation with chlorine dioxide. Although these methods afford a degree of efficacy, none are totally efficient and each presents significant problems for their use. Many of these disinfectants are toxic, and so require special handling by personnel during application. All of these disinfectants leave a residue that must be cleaned up after decontamination. These methods cannot be used when sensitive instruments are present. One example of problems associated with current procedures arose during cleaning of the Hart Office Building in Washington, D.C. following contamination of the building with weapons grade Bacillus anthraxis delivered in the mail stream. Chlorine dioxide gas was the method of choice to mitigate the pathogen. Therefore, the building had to be vacated during and after the treatment until proper ventilation was recorded. The initial treatment was not sufficient and a second application was required.

One of the most recent techniques developed for decontamination of large spaces has involved producing an aqueous vapor generated from mechanical suspension of common disinfectants such as Lysol® or Chlorox® in a mixture with proprietary materials in a commercial atomizer. This vapor is applied to the space to be decontaminated and then ultraviolet wavelength light is used to assure bacterial death. Although somewhat effective, this technique leaves a residue that must be abated and the technique cannot be used to sterilize mail or other contained materials. In addition U.S. Pat. No. 6,436,342 issued to Petri et al. describes chemical compositions and use of sprayable disinfecting compositions for decontaminating surfaces. The preferred embodiment for use of these disinfectant compositions is propulsion via a pressurized propellant.

Decontamination of the mail stream is accomplished by irradiation with gamma irradiation. This requires specialized containment facilities and specially trained personnel for their use. These facilities are expensive and thus cannot be used at all mail sorting and handling sites. During irradiation there is a potential for damage of the mail or its contents as demonstrated by fires set during attempts to decontaminate the mail following the recent anthrax threat.

Little or no effort has been applied to disinfecting large agricultural producing or processing facilities to reduce potential food borne pathogens and to improve the health safety of the workers. These facilities such as brooder houses, livestock barns and slaughterhouses are large and must be affected with animals or workers in place.

No cost effective method of decontaminating large areas from pathogenic fungal growth has been developed. These fungi are becoming and increasing problem in building construction and in container shipping.

Contamination by severely pathogenic organisms or viruses, for example, anthrax spores or smallpox virus, is a significant threat. These pathogens may contaminate surfaces or, worse, be introduced in aerosolized forms that constitute a significant threat to human life. Exposure to these pathogens may result from terrorist activities, from accidental releases, and from the use of biological weapons. Materials used to disinfect surfaces contaminated with bacteria or fungi include chlorine dioxide gas, ethylene oxide, and other highly toxic gasses. These are used, for example, when there is a desire to leave no residue and when sensitive instrumentation is present in the decontamination area. These gasses have some permeability to paper.

Less harmful pathogens may be mitigated by conventional disinfectant liquids and detergents, such as the commercial products Lysol, Pinesol among others, as well as plant oils such as lemon oil among others. Aerosolized combinations of these disinfectant liquids and detergents in water or other solvents may be sprayed onto contaminated surfaces. Increased efficacy may be obtained by using harsher chemicals, such as aqueous dilutions of alcohol, glyceraldehydes and other aldehydes, including formaldehyde and glutraldehyde. In some instances, exposure to ultraviolet light has been used in combination with milder chemical to enhance disinfection.

The use of chemical disinfectants is problematic. Many of these chemicals are irritants or can elicit powerful immune reactions. The gaseous disinfectants used are highly toxic to the user and require special precautions. These are not always effective and often more than a single application is required. When in use the areas being treated must be vacated. The advantage of using these gasses is that they seldom leave residues. Detergents and commercial disinfectants must be applied to contaminated surfaces as liquids and often leave residues that must be cleaned up. Alcohol is only moderately effective against sporulating bacteria and fungi. Glyceraldhyde and formaldehyde are both allerogenic and mutagenic and must be handled with considerable care.

This decade has witnessed many instances of government facilities suffering impaired operations because of pathogens being shipped in the mail, most notably anthrax spores. Decontamination of mail in government facilities is usually accomplished by electron bombardment. This requires that special precautions be taken, such as shielding of the technicians. In practice this has proven to be only modestly effective, has caused fires in paper material, and is expensive to apply. Its usefulness is limited to specialized facilities.

Derivatives of plant oils and fatty acids or lipid derivatives may be used as surface disinfectants and sterilants or as topical, oral or nasal delivered anti microbials or anti virals or as cleansing materials are well known in pharmaceutical uses, in over the counter remedies, in patent medicine and in cleansing solutions. For example, U.S. Pat. No. 6,436,445 issued to Hei et al. describes antimicrobial and antiviral compositions that contain an oxidizing species formed as a reaction product combining a quaternary or protonizable nitrogen compound, an oxidant compound and a halide source. U.S. Pat. No. 6,436,342 issued to Petri et al. describes sprayable disinfecting compositions that contain hydrogen peroxide, an antimicrobial essential oil, and a surfactant system U.S. Pat. No. 5,618,840 issued to Wright describes an antibacterial oil-in-water emulsion that may be used against Helicobacter pylori. An oily discontinuous phase is dispersed in a continuous aqueous phase. The oily discontinuous phase contains an oil carrier and a glycerol ester selected from the group consisting of glycerol monooleate and glycerol monostearate, which may be supplemented by a cationic halogenated hydrocarbon.

Methods of producing methyl soyate and methods of using methyl soyate are well known in the environmental and chemical arts. U.S. Pat. No. 6,281,189 issued to Heimann et al., describes a cleaning composition that may be used for antimicrobial purposes and contains a combination of methyl soyate, d-limonene, and an acidic pH modifying agent. U.S. Pat. No. 6,127,560 issued to Stidham et al. describes a method for preparing a lower alkyl ester product from vegetable oil. In particular, soy oil is extracted and filtered to remove solid fines, and then degummed and bleached. The prepared oil is introduced into a stirred reactor where lower aliphatic monohydric alcohol and an alkaline catalyst is introduced. Alcoholysis proceeds to virtual completion. A lower alkyl alcohol ester phase is separated out and washed with water to remove traces of unreacted alcohol and the alkaline catalyst. The process may be used to produce methyl soyate.

While fumigants such as chlorine dioxide have been employed to decontaminate areas infected with biohazardous microbes, these fumigants leave behind toxic residues that are harmful to people. U.S. Pat. No. 5,635,132 issued to Blanc teaches a method of decontaminating a room. An atomizer is used to diffuse a product containing essential oils as a mist. The essential oils consist essentially of benzoic acid, salol, and thymol; however, the mist is delivered through an atomizer and is therefore very limited in the breadth of deployment and permeability throughout an area.

More recent methods have used a similar atomizer approach in which compositions of commercial disinfectants including such product as Lysol and Chlorox like chlorine based materials are mixed and atomized over a surface followed by exposure to ultraviolet light in the wave length range to kill microorganisms. Like treatment with chlorine disinfectants, this procedure leaves a residue that must be cleaned following exposure.

Very few mechanisms and chemical disinfectants are suitable for use in dispersing chemical disinfectants over large areas. The application of petroleum based oils called “fog oil” to the formation of obscurant smoke for military purposes has been known in the military arts for years; however, the technology has not been converted for decontamination purposes. Methods of generating smoke, together with machines and engines used to generate the smoke, are also well known in the art. Additionally, the art of expelling aerosols has been long known. Methods for producing and expelling aerosols have been variously modified. These technologies are generally not used in the application of disinfectants.

The United Sates Armed Forces use obscurant “fog” or “smoke” to impair the ability of enemy forces to locate materials and personnel in a battlefield situation. Presently this obscurant smoke is generated with petroleum distillate or “fog oil”, a complex mixture of hydrocarbons with properties similar to light weight motor oil. Safety concerns fall around the possibility that fog oil and hence the obscurant smoke generated from it may contain polynuclear aromatic hydrocarbons of PAHs. These compounds are known to be potentially carcinogenic in animal systems. The material used by the U.S. Army is considered to be rendered free of PAH's by severe hydrotreating, however, when analyzed after this treatment PAHs remain present at about 1% level.

One way of generating smoke or aerosol is to use a piezoelectric actuator. For example, U.S. Pat. No. 6,439,474 issued to Denen describes a control system for atomizing liquids with a piezoelectric vibrator where a battery-driven atomizer receives an alternating voltage and applies the same to a piezoelectric actuator that vibrates an atomizing membrane. U.S. Pat. No. 5,803,362 issued to Fraccaroli describes an ultrasonic aerosol apparatus in which a piezoelectric oscillating circuit is temperature-controlled by reducing power input on the basis of sensed temperature to interrupt atomization when temperature is too high and increase power throughput when temperature is too low.

Artificial smoke may be generated by thermo-mechanical devices other than piezoelectric devices. For example, U.S. Pat. No. 5,665,272 issued to Adams et al. describes the use of a multifuel combustion engine to generate obscurant smoke by the action of a Lenoir cycle or constant volume multifuel combustion engine that is capable of operating on gasoline, diesel or kerosene based fuels. U.S. Pat. No. 5,518,179 issued to Humberstone describes an atomizer spraying device using a membrane that is vibrated by a composite thin-walled actuator. U.S. Pat. No. 4,697,520 issued to Brassert et al. describes a fog oil smoke generator that atomizes fog oil by use of a slinger disc that is affixed to and rotates with a turbine wheel to atomize and evaporate the fog oil in a mixture of fog oil and hot turbine gas. U.S. Pat. No. 5,220,637 issued to Levin III, et al. described a method and apparatus for controllably generating smoke in which a smoke-generating fluid is heated within a tubular member to vaporize substantial amounts of the fluid, mix the vaporized fluid with gas flowing upwardly in the tubular member, and produce the smoke.

Even if it were desirable to disperse conventional chemical disinfectants over large areas, such as buildings, industrial complexes, commuter rail lines, or a battlefield, it would be problematic that the chemical disinfectants would themselves constitute hazard to health and safety.

### SUMMARY

The present instrumentalities overcome the problems outlined above and advance the art by providing a safe chemical disinfectant that can be dispersed to decontaminates large areas. The dispersing system may, for example, make use of military smoke generating technology to disperse a transesterified vegetable oil, such as methyl soyate.

Multifold advantages exist in the use of methylated vegetable oils as smoke or aerosol disinfectants. One such advantage is that this procedure leaves no residue and is non-toxic to eukaryotic organisms other than fungi. The smoke generator may be easily constructed, and a variety of such smoke generators are commercially available. Neither the neat oils used to generate the gaseous vapor nor the vapor itself are toxic or mutagenic as tested and shown herein. The vapor usually leaves no residue on the surfaces decontaminated. The gaseous vapor may penetrate paper, and so may be effective in-mail treatment. Technicians may be readily trained to use these methods. The range of spaces that can be decontaminated exceeds that of other methods. The instrumentalities disclosed herein may be safely used in poultry and livestock production facilities and in slaughterhouses. The vapor dissipates rapidly leaving no detectable residue if oil particles are condensed out properly during application.

Relatively inexpensive decontamination of large spaces may be performed with relative ease. By way of example, these spaces include rooms and other indoor spaces contaminated by bioterrorism. The instrumentalities hereof may be use to irradiate sporulating bacteria including species of Bacillus, such as Bacillus anthraxis and Bacillus licheniformis. In poultry and livestock production facilities, decontamination is advantageously obtained of pathogenic bacteria known to enter the food chain including species of Salmonella, Escherichia coli O157H7 and other serotypes, Campylobacter and similar bacteria. It is also possible to decontaminate hospital rooms and surgical suits to improved patient safety. Decontamination of cargo vessels including trucks, airplanes and ships may provide safer transport of goods and prevent terror attacks. Application agricultural crops may be treated to prevent agricultural bioterrorism. Decontamination of public areas including schools and senior citizen areas may eliminate harmful, such as Shigella and Salmonella to provide safer environments for care of infants, children and senior adults. Treatment of entire buildings may ameliorate or eradicate contamination by pathogenic fungi including Penicillium, Aspergillus, Stachybotryus, and others. Decontamination of mail in the mail stream may improve safety for mail handlers and prevent bioterrorism by mail.

Oil aerosols, vapors, or smokes comprising polyaromatic hydrocarbons and fatty acid esters, such as oil aerosols derived from fog oils and methyl soyate are non-mutagenic and nontoxic to test animals such as mice and rats. These materials can be readily produced and deployed over wide areas. As described herein and below, smoke or aerosol forms of methylated vegetable oils may be used to kill microbes. An oil aerosol or smoke exposure-based approach may be used as a method of decontamination of an area and as a bioterrorism counter measure.

Further advantage is found in the low cost of this procedure. It is estimated that maximum cost for a generator is less than $20,000 to clean a room of 1,000 to 3,000 sq. ft. or to decontaminate mail in the mail stream. This is compared to the nearly $1,000,000 to equip a building or facility for cleaning mail with radiation. Also there is no cost or minimal cost to clean up after treatment since this procedure leaves no residue, and hence the cost of the generator would be rapidly recovered. The material for producing the effective vapor will have a cost of $5.00 to $15.00 per gallon and the average decontamination will recover 3-5 gallons. Again this is a significantly lower cost than the currently used procedures.

Transesterified vegetable oils, for example methyl soyate, may not be effective against all fungi forming spores, nor against eukaryotic pathogens such as nematode eggs or protozoa.

### BRIEF DESCRIPTION OF THE DRAWINGS

FIG. 1 depicts a schematic drawing of a representative oil aerosol (smoke) or vapor generator;

FIG. 2 depicts a schematic drawing of a blow-up of the furnace and oil injector of FIG. 1; and

FIG. 3 shows a demise of Salmonella as a function of time exposed to airborne methyl soyate.

### DETAILED DESCRIPTION

The instrumentalities disclosed herein are drawn to systems and methods of decontaminating areas that may contain microbes which are considered to be harmful to plants or animals, especially humans or to have some negative value on the quality of life. The discussion below teaches by way of example, and not by limitation.

Soy oil ester, for example, methyl soyate, smoke and vapor is highly toxic to microbes. This surprising discovery has now culminated in the development of novel applications for fog oil and methyl soyate gaseous vapors as disinfecting and/or sterilizing agents.

Methods of microbial decontamination may be practiced using fog oil or methyl soyate as the basis from which to produce, at high temperatures, a gaseous vapor that acts as an effective sterilant or decontaminate. The fog oil or methyl soyate may be applied to an area or surface as a liquid, smoke, vapor or gas. It is preferred to use a gaseous vapor, which is produced from fog oil or methyl soyate and delivered to an area or surface that may contain a microbe that may be harmful to plants or animals, especially humans. The gaseous vapor results in an effective disinfection of the area or surface without leaving a residue. The area to be decontaminated is fumigated for at least 2 minutes, and more preferably for at least 15 minutes to 30 minutes. Effective decontamination occurs when there is a reduction in the number of microbes that are considered to be harmful in an area or on a surface, wherein the risk of morbid infection or potential infestation of a host is significantly reduced.

By way of example, a decontaminant is formed of a gaseous vapor or an aerosol produced from the condensed vapors of oil. The oil may be petroleum-based, animal-based, plant-based or synthetic. The decontaminant is applied to an area or surface, e.g., by filling the space or area with the gaseous vapor that endures for at least 2 minutes. Alternatively, the gaseous vapor may be applied to a confined area for approximately 5 minutes, then the confined area may be sealed for at least approximately 30 minutes, during which time the aerosolized oil particles and/or gaseous vapor penetrate the area and kill microbes. More preferably the area is subjected to the gaseous vapor or aerosolized oil particles for at least 15 minutes. Under the conditions studied and reported hereinbelow, most strains of bacteria tested were killed after 15 minutes of exposure to the aerosolized oil particles or gaseous vapor, and all strains of bacteria that were tested were killed when subjected to smoke or gaseous vapor for 60 minutes.

Native fog oil and soy oil esters have special antimicrobial properties represented in the aerosol condensates that are collected from the aerosolization process, gaseous vapor that is produced from heating these oils at temperatures greater than 350° C. together with removal of residual moisture. These materials are advantageously non-mutagenic and nontoxic to higher test organisms animals, such as mice and rats, as well as the Drosophila melanogaster, as determined by an X assay (shown below) that was used to detect mutagenic and toxic properties in eukaryotic organisms. On the other hand, the gaseous vapors and aerosol condensates are highly toxic to all bacterial and fungal strains that have been tested to date, and are highly effective in disinfecting surfaces that are contaminated with these microorganisms. The broad-spectrum antimicrobial killing ability of the aerosols and gaseous vapors is enhanced by ready diffusion of vapors through permeable barriers, such as paper and cloth, to sterilize contaminated surfaces behind such barriers. Depending upon the nature of the area to be disinfected or sterilized, exposure times as short as two minutes have been found to be effective. Since the gaseous vapors from the oils can be readily produced and deployed over wide areas, a gaseous vapor that is generated from the oils exposure based approach holds potential as a disinfection technique and a bio-terrorism counter measure.

The many advantages of these instrumentalities at least include: (1) non-toxicity to mammals, including humans; (2) non-mutagenicity; (3) ease of use and application over a wide area or a small confined area; (4) prophylactic films may be applied, especially by aerosol condensation, to leave a non-toxic yet microbiocidal residue; (5) penetration through cloth or paper provides utility as a mail disinfectant or sterilant; (6) administration using art known methods and readily available equipment; and (7) gaseous vaporized particles are capable of distributing completely throughout an area and penetrating small crevices. Due to the broad range of antimicrobial activity toward sporulating bacteria such as Bacillus sp., including Bacillus anthracis, other gram positive bacteria, gram negative bacteria, fungi and viruses, these instrumentalities are also useful to protect against a bioterrorism attack on the home front or on the battle field. Further advantages include use in normal domestic situations, for example, to disinfect large areas in a wide range of environments including agricultural production and processing facilities.

Large areas may be decontaminated or sterilized, especially areas that are at high risk or suspected of being at high risk for harboring microbes considered to be pathogenic to plants or animals, especially humans. Such microbes include, but are not limited to strains of influenza virus, West Nile virus, small pox virus and the like; of virus, bacterial strains such as Bacillus, including Bacillus anthracis, B. lichenformis, B. megaterium, Yersinia, including Yersinia pestis, Salmonella, Escherichia, Shigella, Pseudomonas, Serratia, Enterobacter, Clostridium including Cloastridium botulinum, Campylobacter, Klebsiella, Mycobacterium, Staphylococcus, Bordetella, Streptococcus, Francisella, Legionella, Vibrio, and the like; pathogenic fungi such as Blastomyces, Candida, Stachybotrys, Aspergillus, including Aspergillus candidus, and Aspergillus falvus, Acremonium, Histoplasma, Tinea, Fusarium, including Fusarium solani Ceratocystis, Cladisporium, Penicillium, Botrytis and the like. This list of microbes is exemplary and nonlimiting.

Suitable environments of use include, without limitation, food production facilities, chicken coops and other poultry rearing facilities, bovine and swine rearing and production facilities, laboratories, bio-weapons manufacturing facilities, feed lots, slaughter houses, sewage treatment plants, hospitals and clinics, simulated and real battle fields, ballast tanks of ships, air plane cabins and cargo spaces, kitchens, mail handling facilities, border crossing facilities, schools, and buildings and rooms suspected of or considered to be under biological attack.

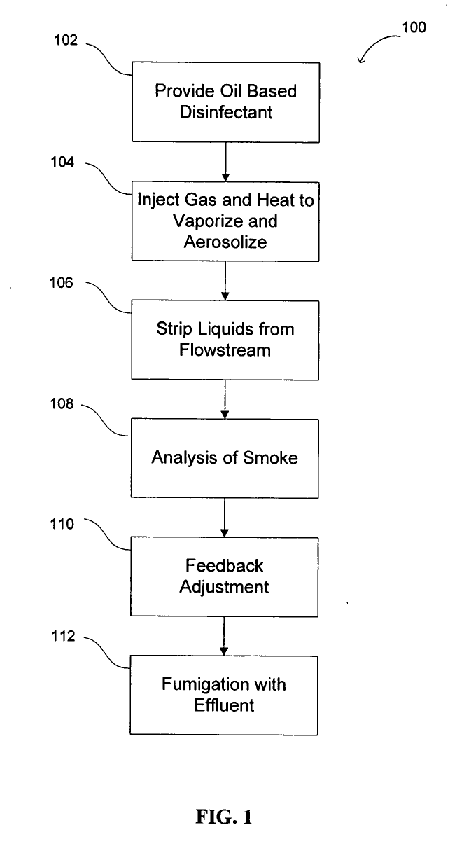

One way of decontaminating is to generate smoke using any apparatus that produces aerosolized oil droplets of the composition herein described and which can be converted to an effective gaseous vapor using a condensation system as depicted. For larger scale gaseous vapor deployment such as on a battle field, at a border crossing, in a slaughter house, or the like, a smoke generator similar in operation to a U.S. Army Generator M-57, which is well known in the art of smoke generation, equipped with condensation apparatus may be employed. For smaller scale gaseous vapor deployment, such as in a laboratory setting, chicken coop, mail room, office space, or the like, a smaller scale smoke generator, such as the one depicted in FIG. 1, may be employed. The skilled artisan in the practice of this invention may substitute smoke generators of various sizes and capacities and modes of operation to accommodate each specific situation to which the gaseous vapor decontaminant may be applied.

The following terms are defined as used herein.

“Methyl soyate” is defined as any ester of oil extracted from soy. The ester is preferably a methyl ester of the formula RCOOCH₃, wherein R may be any hydrocarbon of at least two carbons in length. Methyl soyate may be admixed with additional substances, such as coloring agents, carrier agents, scent agents, and combinations thereof.

“Oil” or “oil mixture” is herein defined as any one of fats, described as fatty acids, phospholipids, sterols, steroids, glycerides, triglycerides, isoprenes, hydrocarbons comprising at least two carbons (including aromatic hydrocarbons), soaps and salts of fatty acids, methyl esters of glycerides, fatty acids (either saturated or unsaturated, and of at least two carbons in length), stearic acids, oleic acids, linoleic acids, linolenic acids, and trans fatty acids, or mixtures thereof, terpenes including mono, di, tri, and poly terpenes, terpenoids, and plant terpenoid resin components including but not inclusive of, such resin components as verbenone, pinene limoene, geranyl and geranol. Oils can be from petroleum, plant, microbial or animal sources.

“Smoke”, as used herein, is defined as a condensate of oil vapors consisting essentially of an aerosol of oil droplets or droplets of chemicals produced during the heating of the oil used in the production of oil vapor. Preferably, an aerosol useful in these instrumentalities, comprises droplets of between 0.15 μm and 1.5 μm in diameter. “Oil aerosol” and “smoke” are used interchangeably.

“Vapor”, as used herein, is defined as the gaseous phase of oils, generally produced by heating the oil at least to its boiling point followed by removal of oil and water residues to produce a gaseous phase which leaves no residue upon condensation.

“Fog oil” is defined as any hydrocarbon used for the purpose of generating obscuring smoke or large white clouds of oil vapor that defeat visual-range observation and tracking methods, such as that used during combat or combat training. Examples of fog oil include diesel, JP4, JP8, MOGAS, Rev III, Rev E, and other petroleum based fuels.

“Microbe”, as used herein, encompasses all prokaryotic organisms, single cell eukaryotic organisms and viruses having DNA or RNA as a genetic information. These include viruses such as the small pox and other vaccinia viruses, geminii viruses of plants among others, plant spores, bacteria, cyanobacteria, yeasts, molds, other fungi, protozoans, and spores thereof.

“Pathogen” as used herein, encompasses all organisms that result in a negative physiological response from a host or agent that it infects or invades. These include viruses, bacteria, and fungi that are known to cause disease or to elicit a negative host response

“Parasite” is defined as any organism which invades and establishes in a host animal or plant and whose function is to derive nutrient from that host without necessarily inducing an obvious pathogenic state but which by its presence decreases the efficiency of that host in performing its natural physiological work.

“Sterilant” or “decontaminant” is defined as an agent of sterilization, wherein the sterilant kills or renders harmless microbes. For the purposes of this body of work the terms “sterilize” and “decontaminate” are equivalent and are used interchangeably.

FIG. 1 shows a decontamination process 100. Step 102 entails providing an oil-based disinfectant or sterilant, such as a methylated vegetable or other suitable oil, oil-based material or mixture. With step 104, the oil-based disinfectant is heated and combined with gas, such as air, nitrogen, or a noble gas, to aerosolize and vaporize oil-based disinfectant in the flowstream. Suitable heating of the oil-based disinfectant for vaporization purposes may include heating oil-based disinfectant to a temperature of at least 350° C. It is possible to assist aerosolization by the action of a venturi, piezoelectric device, or other mechanism for producing aerosols. The formation of aerosol is advantageously utilized with heating to facilitate further vaporization; however, the presence of small droplets in an aerosol mist may be associated with deleterious consequences as condensation of these droplets. Where condensation could be a problem, step 106 is used to strip liquids from the flowstream, for example, by the action of a fibrous filter or centrifugal separator. Accordingly, unvaporized oil and any other particulate materials may be removed from the flowstream via a condensation procedure from which remains only smoke or fog as an effluent gaseous vapor mixed with gas.

Optionally, the smoke or fog may be analyzed by optical or electronic instrumentation in step 108 to ascertain qualities of the smoke or fog. Analytical signals produced in step 108 may be provided as feedback in step 110 to an electronic controller capable of adjusting the quality of smoke or fog by altering the flow rates of oil-based disinfectant, the gas flow rate, or the heating temperature. Fumigation 112 using the smoke or fog results in the killing or rendering of the microbes harmless to animals.

By way of example, the area for decontamination is an area in which the microbe Bacillus anthracis is introduced via an act of terrorism and the animal is a human. To affect disinfection the area is fumigated for at least 15 minutes with a gaseous formed by heating an oil mixture to at least 550° C.

In another example, the area for decontamination is an area in which poultry or live stock for human consumption is produced or prepared and in which bacteria are any and all bacteria, pathogenic to animals particularly humans, upon consumption. These include but are not exclusive to, E. coli, salmolnella spp., Shigella spp., Enterobacter, Camplyobacter, Klebesella, or Helicobacter. To affect disinfection the area is fogged or fumigated for at least 15 min with an aerosol or gaseous vapor produced by heating a plant or light mineral oil to at least 550° C.

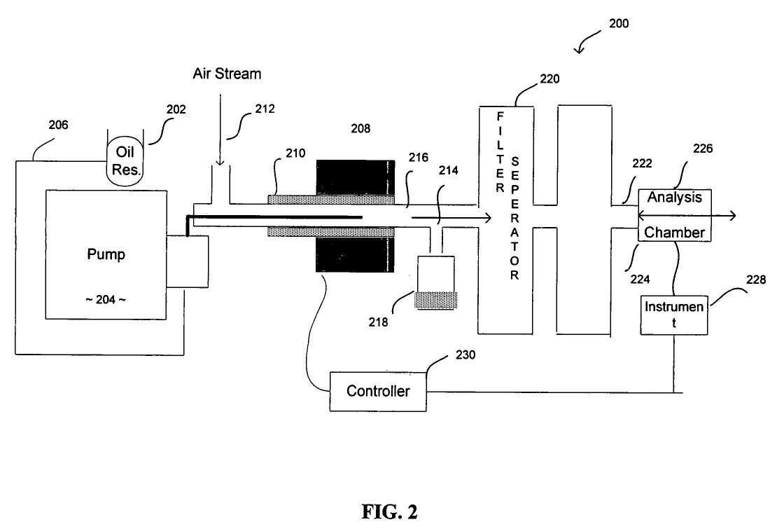

FIG. 2 shows one disinfectant system 200 that may be used to implement the process 100 shown and described in FIG. 1. The disinfectant system 200 is used to decontaminate an average sized room, for example, sized 20 feet by 20 feet by 10 feet, and so the flow rates herein may be adjusted proportionately for larger and smaller areas. An oil reservoir 202 contains a vegetable oil derivative, which is preferably a methylated vegetable oil, and is most preferably methyl soyate. Oil reservoir 202 feeds pump 204 through line 206. Pump 204 may be a reciprocating dual piston pump or another type of pump, for example, one capable of delivering a constant stream of vegetable oil derivative from oil reservoir 202 at a flow rate ranging from 0.1 ml/min to 10.0 ml/min. Pump 204 delivers material from oil reservoir 202 to a thermostatically controlled concentric tubular oven or furnace 206, and electronic heating coil that is electronically controlled to operate at selected temperatures, generally, at any temperature or temperatures ranging from 350° C. to 650° C. (±5° C.). The heating action of furnace 208 at least partially vaporizes the oil-based disinfectant within permeable tube 210 The resulting vapors and, possibly, some liquid droplets, are forced out of permeable tube 210 by an injected air stream 212. The air-flow rate is generally at least about 3 L/min and may suitably range between 3 L/min and 15 L/min. The resulting combined flowstream contains aerosol, mist, and vapor. Larger liquid droplets flowing in line 216 may be collected in a condensed oil collector 218 for eventual removal and disposal. The remaining portions of combined flowstream 214 may pass through a filter separator 220, for example, a dual chambered box packed with glass wool that operates by droplet condensation and agglomeration on the glass wool substrate to further separate vapors from aerosol or mist components in the combined flowstream 214.

The remaining portions of combined flowstream 214 discharge through exit orifice 222 as a smoke or fog 224 that includes air and vapor from the vegetable oil derivative in oil reservoir 202. The filter/separator 220 is optionally omitted for direct discharge of an unfiltered flowstream is droplet condensation is not problematic in the intended environment of use. An analysis chamber 226 is optionally equipped with instrumentation 228 to assess the quality of smoke 224. For example, the instrumentation 228 may be an optical particle counter that provides feedback to a processor-based controller that is programmed with an algorithm to achieve optimal characteristics in smoke 224 by adjusting the temperature of tube furnace 208, the mass flow rate of air stream 212, and/or the output of pump 204. The disinfectant smoke generator system 100 can be operated over a wide range of operational parameters.

In alternative embodiments, the vegetable oil-based disinfectant material within oil reservoir 202 may be admixed with a petroleum-based fog oil, for example, a polyaromatic hydrocarbon selected from naphthalene, phenanthrene, fluoranthrene and pyrene. The petroleum products are substantially all removed in the condensation procedure to produce the gaseous vapor. In another embodiments, the oil-based disinfectant may include methyl soyate that is admixed with phenanthrene, dimethyl phenanathrene, palmitic acid, palmitic methyl ester, stearic acid, stearic acid methyl ester, oleic acid, oleic acid methyl ester, linoleic acid, linoleic acid methyl ester, linolenic acid, and linolenic acid methyl ester, again which are removed in the condensation procedure to produce a gaseous vapor. In another embodiment, the oil-based disinfecant is a plant oil derivative from oil grains, such as flax (linseed oil), corn, sunflower, canola, or palm among others or is derived from plant essential oils including but not exclusive to terpeniols, limenols, geraniols, lemon oil, eucalyptus oil, vanilla bean oils, or from nut seed oils such as walnut oil. These oils may be transesterified in the manner of making methyl soyate.

The area for fumigation may be, for example, a food production facility, poultry rearing facility, cattle or swine rearing or producing facility, bioweapons manufacturing facility, feed lot, slaughter house, sewage treatment plant, hospital, health clinic, battle field, ballast tank of ships, cabins of airplanes, hospital rooms including laboratories and surgery theaters, kitchen, mail handling facility, border crossing facility, school, or the like. The microbe, which is to be killed or rendered harmless, may be, for example, a virus, West Nile virus, influenza virus, small pox virus, bacteria, Bacillus, Yersinia, Salmonella, Escherichia, Shigella, Pseudomonas, Serratia, Enterobacter, Campylobacter, Klebsiella, Mycobacterium, Staphylococcus, Serratia, Bordetella, Streptococcus, Francisella, Legionella, Vibrio, fungi, Candida, Histoplasma, Tinea, or the like.

In one aspect, the area for decontamination may be the subject area of a bioterrorism attack. The area may be indoors or outdoors. The area may be fumigated for at least 30 minutes with a gaseous vapor that is produced by heating fog oil or methyl soyate to at least 350° C., to render harmless remaining vestiges of the bioweapon. Remediation of bioweapons in this manner includes, but is not limited to, those dispersing Yersinia pestis, Bacillus anthracis, Legionella, Penicillium, Bordetella, Fusarium, Aspergillus, Stachybotyris, smallpox virus, Ebola virus, and the like.

It is also possible to use soybean oil in the oil reservoir 202 to generate obscurative smoke. Soybean oil has properties comparable to the petroleum distillate and is advantageously free of PAHs. When heated to high temperature and expressed from a generator as a fog, soy oil produces an aerosol containing particles of approximately 5 μM in mean diameter (range 1-10 μM), which are comparable to those produced by the petroleum distillate produced materials. The soy oil fog is comparable in stability to fog oil produced fog and in fact is a more superior obscurant. Soy oil produced “fog” generates a better particle generation efficiency and the smokes are more stable. In addition the soybean oil-based smokes have broad absorption bands in the infrared light range of the electromagnetic spectrum and interferes with IR detection of objects wishing to be hidden in the smoke. Other vegetable oils show similar properties and may have equal value in producing obscurant.

Although smoke containing aerosols comprising oil particles of any size may be applicable to the practice of this invention, a preferred oil particle size ranges from approximately 0.04 microns to approximately 2 microns in diameter if an aerosol is to be used, however, in the preferred embodiment a gaseous vapor is preferred so as to prevent the deposition of residue.

Smoke is generated by first vaporizing an oil, preferably by heating the oil at approximately 350° C. to approximately 650° C. (±5° C.), followed by condensation of the vaporized oil into aerosol droplets and a gaseous vapor. It is sometimes advantageous to adjust operations of disinfectant system 200, such that aerosol droplets are condensed and the gaseous vapor is dispersed into the air of an area to be decontaminated.

Gaseous vapor that is effective as a sterilant or decontaminant may be produced from any petroleum based fog oil, such as diesel fuel, JP4, JP8, MOGAS or other petroleum based fuels which may be employed as visual obscurants, from vegetable or plant-based oils, from animal fats, or from esterified derivatives thereof, such as soybean oil or methyl esters thereof (“methyl soyate”). Fog oil, smoke and methyl soyate gaseous vapor is effective to kill or arrest the growth of numerous microbes. It has further been demonstrated that the gaseous vapor of the instant invention is able to penetrate paper and effectively kill microbes, thus the gaseous vapor of the instant invention may be employed as a disinfectant/decontaminant of mail suspected of being contaminated with microbes used as biological weapons, such as Bacillus anthracis.

The above disclosure describes several embodiments, which must not be interpreted as limiting the scope of the invention. It is envisioned that the skilled artisan will recognize other embodiments of this invention that are not overtly disclosed herein. The following working examples teach by way of example, and not by limitation, to illustrate preferred materials and methods. The working examples are not to be interpreted as limiting the scope of what is disclosed and claimed.

### EXAMPLE 1

### Method of Generating Smoke

Gaseous vapors of various compositions were generated in a smaller scale (laboratory scale) smoke generator designed to mimic the operation of the U.S. Army Generator M-57. The laboratory scale gaseous vapor generator can be operated over a wide range of operational parameters, which include oil type, oil flow rate, airflow rate and generation temperature. A schematic of the gaseous vapor generation apparatus used in this example is depicted in FIG. 2.

The apparatus used in this example comprises a reciprocating dual piston pump capable of delivering a constant stream of liquid at 0.1-10.0 ml/min. (Model 6000, Waters Associates). The liquid from the pump was delivered to a thermostatically controlled concentric tubular oven, as depicted in FIG. 2. The temperature of the tubular furnace could be controlled over a range of 350° C.-650° C. (±5° C.), and 550° C. was used. The vaporized oil was forced out of the tube with an air stream. The resulting aerosol was fed passed a cold finger condensation apparatus and into a glass-lined monitoring chamber with a stainless steel frame and subjected to a battery of chemical tests and bioassays.

### EXAMPLE 2

### Analysis of Smoke Components

Comparative chemical characterization studies were performed different source oil materials including native soybean oil methyl esters, fog oil, as well as aerosol condensate samples collected from smoke generated as described in Example 1. Selective extraction of samples with dimethyl sulfoxide (DMSO) was performed in combination with elution chromatography on silica gel.

Aliquots of the oil samples were diluted with n-hexane to obtain a 1% solution. A 2 μL portion of each solution was taken and fortified with 500 pg each of five deuterium labeled surrogate aromatics, i.e., 1,4-Dichlorobenzene-d₄, Naphthalene-d₈, Acenaphthene-d₁₀, Phenanthrene-d₁₀, and Chysene-d₁₂. Each fortified sample was extracted twice with 5 mL DMSO. The hexane portion was discarded after the second extraction while the DMSO extracts were pooled. 10 mL of organic free water was added to the pooled DMSO extracts. Each mixture was then back extracted twice with 5 mL of hexane containing 10% benzene. The hexane extract was filtered and “dried” by passing it through a bed of anhydrous sodium sulfate (Na₂SO₄). The Na₂SO₄ bed was rinsed with a 5 mL portion of hexane with 20% n-heptane, which was added to the dried hexane extract. The dried extract was concentrated down to 1 mL under a stream of zero grade nitrogen. A 2 L portion of the concentrated extract was injected into a gas chromatography-mass spectrometery (GC-MS) system, Model 5890 series II and model 5972 (Hewlett-Packard Instruments).

The gas chromatographic separations were carried out with a 30 m×0.25 mm (i.d.) fused silica capillary with surface bound polysiloxane (95% methyl+5% phenyl). Helium was used as the carrier gas under a dynamic pressure control. The linear flow velocity was held constant at 35 cm min⁻¹. The mass spectrometer was operated in selected ion monitoring mode (SIM). Characteristic ions for sixteen condensed ring aromatics and selected akylated aromatics were monitored at selected time windows during the gas chromatographic runs.

The Fog Oil (Rev.E) that was used for during the experiments was obtained from the United States Army Chemical School at Fort Leonard Wood, Mo. Samples obtained from smokes generated at various temperatures, including unheated “native” fog oil, were subjected to gas chromatography-mass spectroscopy (GC-MS). The presence of a number of peaks, in addition to the labeled surrogates and internal standards, were readily observed. The retention time and ion mass-to-charge (m/z) matching revealed that the peaks corresponded primarily to aromatic hydrocarbons such as naphthalene and alkylated aromatics. Higher polyaromatic hydrocarbons (PAHs) such as fluorathene and pyrene were also detected in 5-22 parts per million (ppm) range. The increase in the smoke generation temperature led to a slight but measurable increase in the concentrations of both the condensed ring PAHs and the alkylated PAHs. The increase was most noticeable in the case of phenanthrene whose concentration increased from 6 to 28 ppm. The concentrations of PAHs and the alkylated PAHs detected in the neat Fog Oil and the aerosol condensates are given in Table 1.

TABLE 1
Concentration of PAHs in Fog oil (ppm) (Oil flow rate 0.5 mL min⁻¹
and Air flow rate 10 L min⁻¹)

| Compound name | Native | 350° C. | 400° C. | 450° C. | 500° C. | 550° C. | 600° C. | 650° C. |
|---|---|---|---|---|---|---|---|---|
| PAHs | | | | | | | | |
| Naphthalene (128) | 4 | 4 | 5 | 6 | 6 | 10 | 12 | 15 |
| Acenaphtylene (152) | <0.1 | <0.1 | <0.1 | <0.1 | <0.1 | <0.1 | <0.1 | <0.1 |
| Acenaphthene (153) | <0.1 | <0.1 | <0.1 | <0.1 | <0.1 | <0.1 | <0.1 | <0.1 |
| Fluorene (165) | <0.1 | <0.1 | <0.1 | <0.1 | <0.1 | <0.1 | <0.1 | <0.1 |
| Phenanthrene (178) | 6 | 6 | 9 | 12 | 12 | 18 | 17 | 28 |
| Anthracene (178) | <0.1 | <0.1 | <0.1 | <0.1 | <0.1 | <0.1 | <0.1 | <0.1 |
| Fluoranthene (202) | 5 | 4 | 5 | 4 | 6 | 3 | 3 | 7 |
| Pyrene (202) | 11 | 21 | 28 | 17 | 32 | 18 | 25 | 22 |
| Benzo(a)anthrathene (228) | <0.5 | <0.5 | <0.5 | <0.5 | <0.5 | <0.5 | <0.5 | <0.5 |
| Chrysene (228) | <0.5 | <0.5 | <0.5 | <0.5 | <0.5 | <0.5 | <0.5 | <0.5 |
| Benzo(b)fluoranthene (252) | <0.5 | <0.5 | <0.5 | <0.5 | <0.5 | <0.5 | <0.5 | <0.5 |
| Benzo(k)fluoranthene (252) | <0.5 | <0.5 | <0.5 | <0.5 | <0.5 | <0.5 | <0.5 | <0.5 |
| Benzo(a)pyrene (252) | <0.5 | <0.5 | <0.5 | <0.5 | <0.5 | <0.5 | <0.5 | <0.5 |
| Indeno(1,2,3-cd)pyrene (276) | <0.5 | <0.5 | <0.5 | <0.5 | <0.5 | <0.5 | <0.5 | <0.5 |
| Dibenz (a, h) anthracene (278) | <0.5 | <0.5 | <0.5 | <0.5 | <0.5 | <0.5 | <0.5 | <0.5 |
| Benzo (ghi) perylene (276) | <0.5 | <0.5 | <0.5 | <0.5 | <0.5 | <0.5 | <0.5 | <0.5 |
| Total PAHs | 32 | 43 | 53 | 46 | 64 | 54 | 60 | 72 |
| Methylate PAHs | | | | | | | | |
| 1-Methyl Naphthalene (142) | 1 | 0 | 2 | 1 | 1 | 3 | 2 | 9 |
| 2-Methyl Naphthalene (142) | 1 | 0 | 1 | 1 | 1 | 2 | 2 | 8 |
| 2,6-Dimethyl Naphthalene (156) | 2 | 1 | 2 | 1 | 7 | 4 | 4 | 5 |
| 2,3-Dimethyl Naphthalene (156) | 1 | 1 | 1 | 2 | 2 | 4 | 4 | 7 |
| 1,5-Dimethyl Naphthalene (156) | 1 | 1 | 7 | 0 | 0 | 0 | 0 | 0 |
| 1,2-Dimethyl Naphthalene (156) | 1 | 1 | 1 | 1 | 1 | 2 | 4 | 5 |
| Dimethyl naphthalene | 3 | 1 | 1 | 3 | 4 | 8 | 8 | 16 |
| Dimethyl naphthalene | 2 | 0 | 1 | 2 | 2 | 4 | 6 | 8 |
| 2,3,5-Trimethyl Naphthalene (170) | 1 | 2 | 3 | 4 | 4 | 5 | 7 | 4 |
| Timethyl naphthalene | 3 | 1 | 2 | 3 | 3 | 5 | 4 | 7 |
| Trimethyl naophthalene | 2 | 2 | 2 | 2 | 3 | 5 | 6 | 7 |
| Trimethyl naophthalene | 4 | 1 | 3 | 3 | 4 | 6 | 3 | 7 |
| Trimethyl naophthalene | 1 | 1 | 1 | 2 | 2 | 3 | 2 | 4 |
| 3,6-Dimethyl Phenanthrene (206) | 5 | 6 | 7 | 11 | 8 | 6 | 8 | 10 |
| Dimethyl phenathrene | 7 | 7 | 8 | 11 | 9 | 8 | 8 | 11 |
| Dimethyl phenathrene | 7 | 7 | 8 | 11 | 9 | 10 | 10 | 10 |
| Dimethyl phenathrene | 42 | 40 | 39 | 47 | 44 | 44 | 48 | 52 |
| Dimethyl phenathrene | 14 | 15 | 16 | 20 | 18 | 19 | 17 | 23 |
| Dimethyl phenathrene | 7 | 7 | 8 | 11 | 9 | 9 | 8 | 11 |
| Total Methyl PAHs | 104 | 95 | 115 | 134 | 132 | 147 | 152 | 206 |
| Total | 136 | 137 | 168 | 180 | 196 | 201 | 212 | 278 |

Table 2 provides comparative analytical results that were obtained using methyl soyate.

TABLE 2
Concentration of PAHs in Methyl Soyate (ppm) (Oil flow rate
0.5 mL min⁻¹ and Air flow rate 10 L min⁻)

| Compound name | Native | 350° C. | 400° C. | 450° C. | 500° C. | 550° C. | 600° C. | 650° C. |
|---|---|---|---|---|---|---|---|---|
| PAHs | | | | | | | | |
| Naphthalene (128) | <0.1 | <0.1 | <0.1 | <0.1 | <0.1 | <0.1 | <0.1 | <0.1 |
| Acenaphtylene (152) | <0.1 | <0.1 | <0.1 | <0.1 | <0.1 | <0.1 | <0.1 | <0.1 |
| Acenaphthene (153) | <0.1 | <0.1 | <0.1 | <0.1 | <0.1 | <0.1 | <0.1 | <0.1 |
| Fluorene (165) | <0.1 | <0.1 | <0.1 | <0.1 | <0.1 | <0.1 | <0.1 | <0.1 |
| Phenanthrene (178) | <0.1 | <0.1 | <0.1 | <0.1 | <0.1 | 1 | 6 | 6 |
| Anthracene (178) | <0.1 | <0.1 | <0.1 | <0.1 | <0.1 | <0.1 | <0.1 | <0.1 |
| Fluoranthene (202) | <0.1 | <0.1 | <0.1 | <0.1 | <0.1 | <0.1 | <0.1 | <0.1 |
| Pyrene (202) | <0.5 | <0.5 | <0.5 | <0.5 | <0.5 | <0.5 | <0.5 | <0.5 |
| Benzo (a) anthrathene (228) | <0.5 | <0.5 | <0.5 | <0.5 | <0.5 | <0.5 | <0.5 | <0.5 |
| Chrysene (228) | <0.5 | <0.5 | <0.5 | <0.5 | <0.5 | <0.5 | <0.5 | <0.5 |
| Benzo (b) fluoranthene (252) | <0.5 | <0.5 | <0.5 | <0.5 | <0.5 | <0.5 | <0.5 | <0.5 |
| Benzo (k) fluoranthene (252) | <0.5 | <0.5 | <0.5 | <0.5 | <0.5 | <0.5 | <0.5 | <0.5 |
| Benzo (a) pyrene (252) | <0.5 | <0.5 | <0.5 | <0.5 | <0.5 | <0.5 | <0.5 | <0.5 |
| Indeno (1,2,3-cd) pyrene (276) | <0.5 | <0.5 | <0.5 | <0.5 | <0.5 | <0.5 | <0.5 | <0.5 |
| Dibenz (a, h) anthracene (278) | <0.5 | <0.5 | <0.5 | <0.5 | <0.5 | <0.5 | <0.5 | <0.5 |
| Benzo (ghi) perylene (276 | <0.5 | <0.5 | <0.5 | <0.5 | <0.5 | <0.5 | <0.5 | <0.5 |
| Total PAHs | 0 | 0 | 0 | 0 | 0 | 1 | 6 | 6 |
| Methylate PHAs | | | | | | | | |
| 1-Methyl Naphthalene (142) | <0.1 | <0.1 | <0.1 | <0.1 | <0.1 | <0.1 | <0.1 | <0.1 |
| 2-Methyl Naphthalene (142) | <0.1 | <0.1 | <0.1 | <0.1 | <0.1 | <0.1 | <0.1 | <0.1 |
| 2,6-Dimethyl Naphthalene (156) | <0.1 | <0.1 | <0.1 | <0.1 | <0.1 | <0.1 | <0.1 | <0.1 |
| 2,3-Dimethyl Naphthalene (156) | <0.1 | <0.1 | <0.1 | <0.1 | <0.1 | <0.1 | <0.1 | <0.1 |
| 1,5-Dimethyl Naphthalene (156) | <0.1 | <0.1 | <0.1 | <0.1 | <0.1 | <0.1 | <0.1 | <0.1 |
| 1,2-Dimethyl Naphthalene (156) | <0.1 | <0.1 | <0.1 | <0.1 | <0.1 | <0.1 | <0.1 | <0.1 |
| Dimethyl naphthalene | <0.1 | <0.1 | <0.1 | <0.1 | <0.1 | <0.1 | <0.1 | <0.1 |
| Demethyl naphthalene | <0.1 | <0.1 | <0.1 | <0.1 | <0.1 | <0.1 | <0.1 | <0.1 |
| 2,3,5-Trimethyl Naphthalene (170) | <02 | <02 | <02 | <02 | <02 | <02 | <02 | <02 |
| Trimethyl naphthalene | <0.2 | <0.2 | <0.2 | <0.2 | <0.2 | <0.2 | <0.2 | <0.2 |
| Trimethyl naphthalene | <0.2 | <0.2 | <0.2 | <0.2 | <0.2 | <0.2 | <0.2 | <0.2 |
| Trimethyl naphthalene | <0.2 | <0.2 | <0.2 | <0.2 | <0.2 | <0.2 | <0.2 | <0.2 |
| Trimethyl naphthalene | <0.2 | <0.2 | <0.2 | <0.2 | <0.2 | <0.2 | <0.2 | <0.2 |
| 3,6-Dimethyl Phenanthrene (206) | <0.5 | <0.5 | <0.5 | <0.5 | <0.5 | <0.5 | <0.5 | <0.5 |
| Dimethyl phenanthrene | <0.5 | <0.5 | <0.5 | <0.5 | <0.5 | <0.5 | <0.5 | <0.5 |
| Dimethyl phenanthrene | 0.5 | 0.5 | 0.5 | 0.5 | 0.5 | 0.5 | 0.5 | 0.5 |
| Dimethyl phenanthrene | <0.5 | <0.5 | <0.5 | <0.5 | <0.5 | <0.5 | <0.5 | <0.5 |
| Dimethyl phenanthrene | <0.5 | <0.5 | <0.5 | <0.5 | <0.5 | <0.5 | <0.5 | <0.5 |
| Dimethyl phenanthrene | <0.5 | <0.5 | <0.5 | <0.5 | <0.5 | <0.5 | <0.5 | <0.5 |
| Total Methyl PAHs | 0 | 0 | 0 | 0 | 0 | 0 | 0 | 0 |
| Total | 0 | 0 | 0 | 0 | 0 | 1 | 6 | 6 |

In contrast to the Fog oil, the native methyl soyate was substantially devoid of PAHs. The primary constituents of this oil are the five fatty acid methyl esters, i.e., palmitic acid methyl ester, strearic acid methyl ester, oleic acid methyl ester, linoleic acid methyl ester, and linolenic acid methyl ester. The only peaks observed in the total ion chromatogram were those of the surrogates and the internal standard. The concentrations of PAHs in the neat methyl soyate and aerosol condensate of methyl soyate that were obtained at different generation temperatures are given in Table 2. The only change in PAHs that were observed was for phenathrene, which increased in concentration from <0.1 to 6 ppm.

The number of aerosol particles produced per unit volume of oil introduced is referred to herein as a particle density. This value was monitored in the laboratory as a measure of aerosol generation efficiency. Generation efficiency was measured at oil flow rates of 0.25 to 1.5 mL min⁻¹, airflow rates of 3-15 L min⁻¹ and generation temperatures of 500-600° C. The experimental set-up used for these measurements consisted of a dilution chamber, a bipolar charger, a dynamic mobility analyzer (DMA) and a condensation nuclei counter (CNC). The Aerosol laden air stream from the generator was diluted with an auxiliary air stream of 236 L min⁻¹ prior to its introduction into the DMA and CNC assembly. The sample gas flow rate through the DMA was maintained at 3 L min⁻¹. The results of the aerosol efficiency experiments carried out with Methyl soyate and Fog oil at an oil flow of 1 mL min⁻¹ are shown in Tables 3 and 4, respectively.

TABLE 3
Number density and Size Distribution of Methyl Soyate Aerosol*

| | T 500 Air* | | | | T 550 Air | | | | Air | | | |
|---|---|---|---|---|---|---|---|---|---|---|---|---|
| Size | ° C. (L/min) | | | | ° C. (L/min) | | | | T 600° C. (L/min) | | | |
| (um) | 3** | 5** | 10** | 15** | 3** | 5** | 10** | 15** | 3** | 5** | 10** | 15** |
| 2.043 | 250 | 350 | 60000 | 100 | 200 | 3500 | 4000 | 4000 | 100 | 180 | 7000 | 4000 |
| 1.09 | 7500 | 50000 | >99000 | 2300 | 6000 | 15000 | 28000 | 13000 | 3000 | 4600 | 35000 | 29000 |
| 0.604 | 25000 | 60000 | >99000 | 3500 | 45000 | 48000 | <99000 | 50000 | 28000 | <99000 | <99000 | 40000 |
| 0.352 | 75000 | 90000 | >99000 | 6000 | 90000 | >99000 | >99000 | 69000 | 90000 | >99000 | >99000 | >99000 |
| | >99,00 | | | | | | | | >9900 | | | |
| 0.216 | 0 | >99000 | >99000 | 8200 | 85000 | >90000 | >99000 | 90000 | 0 | >99000 | >99000 | >99000 |
| 0.139 | 90000 | 46000 | >99000 | 800 | 8500 | >99000 | >99000 | 35000 | 30000 | 10000 | >99000 | >99000 |
| 0.092 | 3500 | 7500 | 10000 | 60 | 5000 | 9500 | 12000 | 4000 | 1700 | 200 | 1400 | 3000 |
| 0.063 | 400 | 850 | 1700 | 30 | 800 | 3000 | 3500 | 2500 | 90 | 50 | 500 | 200 |
| 0.043 | 10 | 24 | 55 | 20 | 20 | 20 | 10 | 10 | 12 | 10 | 20 | 10 |

*Methyl soyate flow rate was set at 1 mL min⁻¹, number density expressed in particles cc⁻¹

TABLE 4
Number density and Size Distribution of Fog Oil Aerosol*

| | T Air* | | | | T Air | | | T Air | | | | |
|---|---|---|---|---|---|---|---|---|---|---|---|---|
| Size | 500° C. (L/min) | | | | 550° C. (L/min) | | | 600° C. (L/min) | | | | |
| (um) | 3** | 5** | 10** | 15** | 3** | 5** | 10** | 15** | 3** | 5** | 10** | 15** |
| 2.043 | 600 | 1200 | 50000 | 3000 | 100 | 900 | >99000 | 1000 | 300 | 1700 | 9000 | 320 |
| 1.09 | 12000 | 22000 | 99000 | 14000 | 14000 | 9000 | >99000 | 11000 | 7000 | 7500 | 25000 | 590 |
| 0.604 | 15000 | 47000 | >99000 | 52000 | 28000 | 14000 | >99000 | 44000 | 28000 | 14000 | 60000 | 1000 |
| 0.352 | 12000 | 22000 | >99000 | >99000 | 50000 | 63000 | >99000 | >99000 | 65000 | 19000 | >99000 | 7500 |
| 0.216 | 57000 | 10000 | >99000 | >99000 | 23000 | 25000 | >99000 | >99000 | 48000 | 13000 | >99000 | 8500 |
| 0.139 | 2800 | 5400 | >99000 | 65000 | 4500 | 6500 | >99000 | 28000 | 1200 | 7000 | 25000 | 480 |
| 0.092 | 230 | 680 | 3500 | 2000 | 3600 | 4000 | 6000 | 200 | 300 | 150 | 200 | 30 |
| 0.063 | 150 | 400 | 120 | 150 | 120 | 180 | 240 | 150 | 100 | 400 | 66 | 2 |
| 0.043 | 12 | | 14 | 2 | 10 | 25 | 23 | 15 | 20 | 28 | 1 | 2 |

*Fog oil flow rate was set at 1 mL min⁻¹, number density expressed in particles cc⁻¹

The results obtained at 1 mL min⁻1 and other oil flow rates showed a similar trend with both fog oil and methyl soyate. The particle densities obtained were very similar. Methyl soyate yielded higher number densities at 550° C. and 600° C., while fog oil gave slightly higher densities at 500° C. The differences most likely stem from the differences in the boiling points and the flash points of the two oils. The boiling point and flash points for methyl soyate are slightly higher than those of the fog oil. No clear difference in aerosol generation efficiency and air flow rate was observed, except that higher air flow rates (i.e., at least 15 L/min) led to lower generator temperature resulting in a loss in generation efficiency.

Particle size distribution was also monitored. Experiments have shown that particles between 0.1 and 1 micron possess relatively long lifetime in air under ambient conditions. Thus, particles with a mean diameter of 0.3 to 0.5 micron are most desirable for maintaining long lifetime in air under ambient conditions. The overall size distribution of aerosol and number densities obtained with methyl soyate and fog oil were very similar. The size distribution for both types of aerosol was in the range of 0.04 to 2 microns, with a modal range of from approximately 0.15 to approximately 1.5 microns.

### EXAMPLE 3

### Demonstration of Antimicrobial Properties of Oil Aerosols (Smoke)

Fog oil and methyl soyate aerosols were tested for their toxicity toward various microbes. The oil aerosols used in this study were generated through a process that mimics the operation of a “fog oil smoke” generator used by the United States Army. The process involves volatilization and subsequent condensation of fog oils and soy oil esters yielding particles of approximately 0.5-1 micron in diameter, which are effective obscurants for visible light and stabile in ambient air for up to 30 minutes.

Plates containing enriched nutrient minimal agar (MNA) media were inoculated with Salmonella strains, placed into an exposure chamber, and exposed to fog oil or methyl soyate smokes for a duration ranging from approximately 30 seconds to 2 minutes. The smokes (oil aerosols) were generated by introducing 0.5 mL min⁻1 of oil into a stainless steel tube maintained at 350° C. Controls were plates containing MNA media that were placed in an exposure chamber for 30 minutes in the absence of exposure to oil aerosol. After exposure all plates were incubated at 37° C. for 24 hours and examined for the presence of Salmonella colonies. Examination of the MNA plates exposed to aerosols for various intervals of time showed that Salmonella colonies were present only on plates that were exposed to oil aerosol for less than 2 minutes. No colonies were observed on those plates that were exposed for longer time intervals, demonstrating that oil aerosol exhibits high toxicity towards Salmonella.

In a variation of the above experiment, MNA plates were covered with ordinary paper or paper tissues and exposed to oil aerosol (fog oil or methyl soyate) for 2 minutes or 5 minutes. The plates were removed from the chamber, inoculated with the bacterial cultures, incubated for 24 hours, and examined for the presence of bacterial colonies. No colonies grew, demonstrating that the microbiocidal agent found in oil aerosols is able to penetrate paper while maintaining its effectiveness as a microbiocide. In a variation of this paper experiment, examination of paper and paper tissue exposed to oil aerosol prior to inoculation plates gave results similar to those obtained with direct exposure plates. No Salmonella colonies were observed on plates exposed for intervals longer than two minutes. This result indicates that a microbiocidal agent produced during aerosol generation of fog oil or methyl soyate readily diffuses through paper or wipe tissue, gets incorporated into the agar matrix, and makes the agar unfit for microbial growth.

In yet another variation of the above experiment, MNA plates were exposed to oil aerosols (fog oil or methyl soyate) and, after removal from the chamber, placed in a sterile hood for a period of from 5 to 30 minutes. The plates were then inoculated with the bacterial culture, incubated for 24 hours, and examined for the presence of bacterial colonies. Examination of plates exposed to oil aerosol prior to inoculation gave results similar to those obtained with direct exposure plates. No Salmonella colonies were observed on plates exposed for intervals longer than two minutes. This result demonstrates that the microbiocidal agent produced during oil aerosol generation is incorporated into the agar matrix and makes agar unfit for bacterial growth.

Additional exposure experiments were carried out to assess the toxicity of fog oil and methyl soyate aerosol toward a wider range of microbial strains. The exposure experiments were repeated with MNA plates that were preinoculated with the organisms listed in Table 5 and exposed to fog oil and methyl soyate “smokes” for 2 minutes. After the exposure the plates were incubated for 48 hours at 37° C. and examined for the presence of microbial colonies. The results are summarized in Table 5. A plus sign (+) signifies microbial growth. A minus sign (−) signifies no growth or microbial death. No microbial colonies were observed in any of the plates exposed to the fog oil smoke, whereas some colonies were observed on Pseudomonas aeruginose plates exposed to methyl soyate aerosols.

TABLE 5
Microbes Tested for Toxicity of Fog Oil and Methyl Soyate
Aerosols for 2 minutes

| Organism Used | Fog oil | Methyl soyate |
|---|---|---|
| Salmonella typhimurium | − | − |
| Klebsiella pneumonia | − | − |
| Escherichia coli 25922 | − | − |
| Pseudomonas aeruginosa | − | + |
| Enterobacter cloacae | − | − |
| Serratia marcescens | − | − |

MNA-containing petri dishses were inoculated with bacterial species and incubated for 24 hours. The petri plates were then exposed to fog oil aerosol for 15 minutes, 30 minutes, or 60 minutes. The fog oil aerosol (smoke) was generated under the following parameters: oil flow rate at 0.5 mL min⁻¹; oil type was Fog Oil (Rev E.); air flow at 10 L min⁻¹; aerosol generation temperature at 650° C.

After the exposure of the bacterial colonies to the oil aerosol, isolated colonies were transferred from the Petri dishes into 0.5 mL of nutrient broth. Negative controls were nutrient broth with no bacteria. Positive controls comprised the introduction of known bacteria species into nutrient broth.

Strains of fungi, such as Aspergillus, Candida, Histoplasma, Cryptococcus, Neurosporra, Saccharomyces, and the like may be tested for susceptibility to the sterilizing effects of oil aerosol in a similar manner as shown herein for bacteria. Strains of viruses, such as HIV, West Nile, Influenza, Small Pox, and the like may also be tested for susceptibility to the sterilizing effects of oil aerosol in a similar manner as shown herein for bacteria.

Tables 6-8 summarize the results of the oil aerosol exposure. Table 6 shows bacterial survivability after a 15 minute exposure, as indicated by turbidity (growth) in the nutrient solution. The plus (+) sign indicates growth where as the minus (−) sign indicates no growth or bacterial death. The results indicate that the 15 minute exposure regimen is sufficient to kill or arrest bacterial species such as Bacillus megaterium, Bacillus subtilis, Staphylococcus aureus and Pseudomonas aeruginos. However, other species Klebsiella pneumoniae, Enterobacter cloacae, Serratia marcescens, Shigella flexneri, Salmonella typhimurium and Escherichia coli can survive the 15 minute exposure. Table 7 shows bacterial survivability after a 30 minute exposure to oil aerosols. This exposure was toxic to nearly all microbial species included in the test. Only Shigella flexneri survived the 30 minute exposure to oil aerosols. A 60 minute exposure to oil aerosols was shown to be lethal to all microbial species tested, see Table 8.

TABLE 6
Oil aerosol exposure duration: 15 minutes

| Bacteria | 1 | 2 | 3 | 4 | 5 | 6 | 7 | 8 |
|---|---|---|---|---|---|---|---|---|
| Bacillus megaterium | − | − | − | − | − | − | − | − |
| Bacillus subtilis | − | − | − | − | − | − | − | − |
| Klebsiella pneumoniae | + | + | + | + | − | − | − | − |
| Enterobacter cloacae | + | − | + | + | − | + | + | − |
| Serratia marcescens | + | + | − | − | + | − | − | − |
| Staphylococcus aureus | − | − | − | − | − | − | − | + |
| Pseudomonas aeruginosa | + | − | − | − | − | − | − | − |
| Shigella flexneri | + | + | + | + | + | + | + | − |
| Salmonella typhimurium | + | + | + | + | + | + | + | + |
| Escherichia coli | − | + | + | − | + | + | + | + |

TABLE 7
Oil aerosol exposure duration: 30 minutes

| Bacteria | 1 | 2 | 3 | 4 | 5 | 6 | 7 | 8 |
|---|---|---|---|---|---|---|---|---|
| Bacillus megaterium | − | − | − | − | − | − | − | − |
| Bacillus subtilis | − | − | − | − | − | − | − | − |
| Klebsiella pneumoniae | − | − | − | + | − | − | − | − |
| Enterobacter cloacae | − | − | − | − | − | − | − | − |
| Serratia marcescens | − | − | + | − | + | − | − | − |
| Staphylococcus aureus | − | − | − | − | − | − | − | − |
| Pseudomonas aeruginosa | − | + | − | + | − | − | − | − |
| Shigella flexneri | + | + | + | + | + | + | + | − |
| Salmonella typhimurium | − | − | − | − | − | − | − | − |
| Escherichia coli | − | − | − | − | − | − | − | − |

TABLE 8
Oil aerosol exposure duration: 60 minutes

| Bacteria | 1 | 2 | 3 | 4 | 5 | 6 | 7 | 8 |
|---|---|---|---|---|---|---|---|---|
| Bacillus megaterium | − | − | − | − | − | − | − | − |
| Bacillus subtilis | − | − | − | − | − | − | − | − |
| Klebsiella pneumoniae | − | − | − | − | − | − | − | − |
| Enterobacter cloacae | − | − | − | − | − | − | − | − |
| Serratia marcescens | − | − | − | − | − | − | − | − |
| Staphylococcus aureus | − | − | − | − | − | − | − | − |
| Pseudomonas aeruginosa | − | − | − | − | − | − | − | − |
| Shigella flexneri | − | − | − | − | − | − | − | − |
| Salmonella typhimurium | − | − | − | − | − | − | − | − |
| Escherichia coli | − | − | − | − | − | − | − | − |

Additional experiments were performed to test whether a continuous generation of oil aerosols is required for the oil aerosols to be effective in the decontamination of areas containing microbes. The following parameters were employed in the generation of smoke in these experiments: oil flow rate at 0.5 mL min⁻¹, oil type was fog oil, air flow at 10 L min⁻¹, aerosol generation temperature at 650° C.

The oil aerosol was generated and administered into a chamber for a period of 5 minutes. The chamber, which contains plates inoculated with 24 hr incubated bacteria and plates inoculated with sporulating bacteria, was completely sealed after the administration of the oil aerosol. The plates were left inside the chamber for a period of 30 minutes or 60 minutes. The MNA plates were then incubated for an additional 24 hrs at 37° C. The cultures of sporulating bacteria (e.b., Bacillus and other strains) were transferred to a sterile hood and inoculated in trypticase soy broth. The tubes were then incubated in a shaker that was maintained at 37° C. All of the microbes tested failed to grow under these experimental conditions. The results are presented in Table 9. A plus sign (+) indicates growth of cultures. A minus sign (−) indicates no growth of cultures.

TABLE 9
Growth of cultures after 5 minutes of smoke administration
followed by incubation in a sealed chamber for the indicated time.

| Bacteria | Control | 30 min | 60 min |
|---|---|---|---|
| Shigella flexneri | + | − | − |
| Klebsiella pneumoniae | + | − | − |
| Bacillus megaterium | + | − | − |
| Pseudomonas aeruginosa | + | − | − |
| Enterobacter cloacae | + | − | − |
| Staphylococcus aureus | + | − | − |
| Escherichia coli | + | − | − |
| Salmonella typhimurium | + | − | − |
| Serratia marcescens | + | − | − |

### EXAMPLE 4

### Lack of Mutagenicity in Drosphilia

Mutations produced on the X-chromosome of Drosophila melanogaster can be identified easily by mutagenizing male flies and crossing them to compound −X, C(1) females. These females have both of their X-chromosomes attached to the same centromere. This means that both X-chromosomes will be inherited as a unit rather than segregating from each other during meiosis I. This means that female progeny will get both of their X-chromosomes from their mother and, importantly, male progeny will get their X-chromosomes from their father. This means that any X-linked mutation produced in sperm of the father will be passed on to the sons.

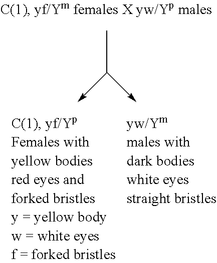

All mutations are X-linked. Y^{m} is the Y chromosome of the mother. Y^{p} is the Y-chromosome of the father. In this cross the male progeny get their X-chromosome from their father and their Y chromosome from their mother. This is just the opposite of normal sex-linked inheritance.

The production of sex-linked lethal mutations is an effective way to monitor for the presence of chemical mutagens. For example ethylmethane sulfonate (EMS) has been shown to produce over 40% sex linked lethals in Drosophila melanogaster exposed to this chemical (Ohnishi, 1977; Genetics 87: 519-527). This high-rate of mutagenesis can be seen as a reduction in the ratio of females to males, since males carrying an X-chromosome with a lethal mutation will die. There will then be a reduction of the male progeny relative to female progeny. If the ratio of females to males is significantly higher in treated as compared to untreated males in matings, then it can be concluded that the treatment caused a significant number of lethal mutations on the treated X-chrosomes.

Yw/Y^{p} males were exposed to the gaseous vapor generated by heating methyl soyate to 650 C for 15 or 30 minutes. A control of C(1), yf/Y females X yw/Y untreated males was included in the analysis. The exposed males were mated with virgin C(1), yf/Y females using standard procedures in glass vials containing fly food medium. After sufficient time for mating, the parents were removed and the number of males and females were counted as the eggs hatched. Table 10 reports the results.

TABLE 10
X-Mutaton Study Results

| | | | Ratio |
|---|---|---|---|
| Treatment/Breeding | Male | Female | (M:F) |
| C(1) y f/Y females x yw/Y untreated males | 875 | 444 | 1.971 |
| C(1) y f/Y females x yw/Y males w/15 min | 974 | 453 | 2.150 |
| exposure | | | |
| C(1) y f/Y females x yw/Y males w/30 min | 879 | 430 | 2.044 |
| exposure | | | |

No significant proportionality differences were observed in the number of males compared to the number of females in those matings in which the males were exposed to the disinfectant gaseous vapor as compared to control matings were observed. Thus it can be concluded that the gaseous vapor is non mutagenic in a standard test for mutagenesis.

### EXAMPLE 5

### Mutagenic Potential of Oils Used to Generate Smoke

The mutagenicity of the native oils generated in the laboratory was evaluated using the Ames test. The toxicity was determined by the frequency of mutations resulting from exposure of Salmonella typhimurium to both types of oil.

The modified Ames test is designed to determine the mutagenicity of compounds by detecting reversion mutations in a series of Salmonella typhimurium strains, each with a different type of mutation. Each S. typhimurium strain contains a point mutation within a gene required for histidine biosynthesis. Therefore, these strains require histidine as a growth factor when grown on minimal glucose medium. Exposure of each S. typhimurium strain to a compound that can cause mutations can lead to reversal of the point mutation that enables the strain to grow on minimal glucose medium in the absence of histidine. The number of bacteria that revert to growth in the absence of histidine correlates with the mutagenicity of the compound. Not all mutagenic compounds cause the same types of mutations; therefore, it is necessary to use strains of S. typhimurium containing different types of point mutations in order to detect all possible mutagens. In addition, some chemicals are not mutagenic until they enter the body and are processed by the liver. Therefore, it is important to also test the compounds in the presence of liver extract that mimics the situation encountered within the body.

Ames strains TA97, TA98, TA100 and TA102 available in the public domain were used. The sensitivity of each strain to chemical mutagens known to cause a high frequency of mutations was used as a control. The control and test compounds were done in both plate incorporation assays and in disk diffusion assays.

For the plate incorporation assay, the bacteria and the control or test compound were combined together in top agar that then was spread onto the surface of a minimal glucose agar plate. For the disk diffusion assay S. typhimurium strains were inoculated into top and the top agar spread on the surface of a minimal glucose agar plate then a sterile filter paper disk saturated with the control or test compound was applied to the surface of the solidified top agar overlay.

The disk diffusion assay is that it is easier to perform and it provides exposure to a wide range of mutagen concentrations as the mutagen diffuses away from the filter disk. This is important because at high mutagen levels, the mutagen is often lethal (observed as a clear zone surrounding the filter disk), and at low concentrations the mutagen does not cause a high enough frequency of mutations to be detected above the background reversion frequency. The plate incorporation assay has the advantage of being more quantitative but requires that several concentrations be tested to find the concentration that is not lethal to the test organisms but causes a high enough frequency of mutations to be detected.

Mutagenicity of native methyl soyate and fog oil and residues from the oil aerosol was tested in both plate incorporation and filter disk assays using each of the four test strains. Oils used in a direct plate assay were the neat methyl soyate, neat fog oil (Rev. E) and condensed oils collected at the end of the generation tube. Water or a commercial baby oil sample (Johnson and Johnson) served as the control. In an oil-spreading assay, oil samples were dissolved in acetone or DMSO (125 μL in 1.5 mL) and a 250 aliquot of the solution was added to the Minimal Glucose Medium (MGM) plate top agar. The top agar was then inoculated with 100 μL of culture containing all four strains of the Salmonella. The plates were incubated for 3 days and examined under a microscope for the presence of Salmonella colonies. A 25 μL aliquot of the oil samples was spread over the top agar with glass beads. The plates were then inoculated with the culture and incubated for 3 days. The plates were examined under a microscope for the presence of Salmonella colonies. A disc plate assay was performed using sterilized filter discs (2 cm diameter) that were exposed to oil aerosols obtained at different temperatures and then placed on the inoculated MGM plates and incubated for 3 days. The plates were then examined under a microscope for the presence of Salmonella colonies.

Table 11 provides the results from the solution incorporation assay for the Fog oil and the Methyl soyate are given in Table VI. Acetone was used as the solvent and water was used as the control. The assay was repeated several times to get consistent colony counts.

TABLE 11
Ames Assay Results for “Native” Methyl Soyate and Fog Oil

| Samples | TA97 | TA98 | TA100 |
|---|---|---|---|
| Control | 120 colonies | 19 colonies | 100 colonies |
| Fog oil | 135 colonies | 30 colonies | 140 colonies |
| Methyl soyate | 127 colonies | 24 colonies | 135 colonies |

There was no significant proportional difference in mutagenic rate between control and experimental treatments indicating that both neat oils are non-mutagenic.

The solution incorporation assay was repeated with condensed oils. The oils were collected during aerosol generation at 350, 450 and 550° C. A 50 μL aliquot of the oils was dissolved in 1.0 mL of DMSO. A 250 μL portion was added to the prior to inoculation and the three-day incubation. The Johnson & Johnson baby oil was used as the control. The results for Methyl soyate are summarized in Table 12.

TABLE 12
Ames Assay Results for Methyl Soyate Condensates

| Strain | Control | 350° | 450° | 550° |
|---|---|---|---|---|
| TA97 | 80 colonies | 118 colonies | 110 colonies | 110 colonies |
| TA98 | 30 colonies | 25 colonies | 30 colonies | 30 colonies |
| TA100 | 100 colonies | 85 colonies | 100 colonies | 125 colonies |

The number of colonies observed for the baby oil and the condensed methyl were low and indicated no mutagenic activity for these oils. The results obtained from similar assay performed with the condensed Fog oil are given in Table 13.

TABLE 13
Ames Assay Results for Fog oil Condensates

| Strain | Control | 350° | 450° | 550° |
|---|---|---|---|---|
| TA97 | 130 colonies | 105 colonies | 120 colonies | 115 colonies |
| TA98 | 25 colonies | 30 colonies | 20 colonies | 20 colonies |
| TA100 | 125 colonies | 140 colonies | 145 colonies | 140 colonies |

The number of colonies observed for the baby oil and the condensed Fog oils were low and indicated no mutagenic activity for these oils

To verify the that the solvents used in the above assays had no effect on the mutagenic rate of Salmonella strains, a thin layer of oil was deposited directly onto top agar with clean sterilized borosilicate glass beads. Baby oil (Johnson & Johnson) was used as the control. The results are not statistically different and show that both methyl soyate and fog oil are not mutagenic.

TABLE 14
Ames Assay Results for Neat Methyl Soyate and Fog oil

| | Strain | | |
|---|---|---|---|
| | TA97 | TA98 | TA100 |
| Control | 120 colonies | 18 colonies | 150 colonies |
| Methyl soyate | 85 colonies | 23 colonies | 125 colonies |
| Fog oil | 130 colonies | 23 colonies | 130 colonies |

The results obtained with Methyl soyate aerosols generated at 350, 450 and 550° C. are given in Table 15.

TABLE 15
Ames Assay Results for Fox oil Aerosol

| Strain | Control | 350° | 450° | 550° |
|---|---|---|---|---|
| TA97 | 100 colonies | 115 colonies | 85 colonies | 110 colonies |
| TA98 | 140 colonies | 30 colonies | 30 colonies | 36 colonies |
| TA100 | 125 colonies | 155 colonies | 120 colonies | 125 colonies |

The results obtained with Fog oil aerosols generated at 350, 450 and 550° C. are given in Tables 16.

TABLE 16
Ames Assay Results for Methyl Soyate Aerosol

| Strain | Control | 350° | 450° | 550° |
|---|---|---|---|---|
| TA97 | 50 colonies | 30 colonies | 35 colonies | 25 colonies |
| TA98 | 20 colonies | 15 colonies | 10 colonies | 13 colonies |
| TA100 | 110 colonies | 85 colonies | 20 colonies | 15 colonies |

### EXAMPLE 6

### Food Processing Environment

A series of tests were carried out to confirm the toxicity of oil vapor on Salmonella strains under different exposure regimes. Exposure time indicates the continuous fumigation of the oil vapor from the generator. All the exposure to the vapor was performed in the 4 cubic feet size chamber with ambient room temperature of 20° C. to 25° C. and humidity between 25% and 60%.

T-soy agar plates were inoculated with 20 μl of overnight grown Salmonella culture (OD ranging from 0.2 to 0.6) containing approximately 10⁷ cells, and exposed to mineral oil or methyl soyate vapor for 30 minutes. Plates were removed from the exposure chamber and incubated for 24 hours at 37° C. No colonies were observed on plates exposed. Control plates that were not exposed to the vapor showed growth.

T-soy agar plates were inoculated with Salmonella and incubated for overnight and then exposed to the vapor for 30 minutes and one hour respectively. Plates were removed from the exposure chamber and colonies on the plates were transferred to the new T-soy agar plate and incubated for 24 hours at 37° C. Colonies from the plates exposed to the vapor for one hour did not grow back, however, the 30 minutes exposed one did grow back.

T-soy agar plates were exposed to the vapor of mineral oil or methyl soyate for 30 minutes. After exposure, Salmonella was inoculated and incubated for 24 hours at 37° C. No Salmonella colonies were observed on plates exposed. This experiment showed that toxic agent produced during the vapor generation is incorporated into the agar matrix and makes agar unfit for bacterial growth.

### EXAMPLE 7

### Broad Spectrum of Antimicrobial Efficacy

The efficacy of the oil vapor has been tested against a broad range of microorganisms. Table 17 shows a representative listing of organisms tested. The classes of organisms are also shown. Nutrient agar plates were inoculated with different bacteria and exposed to the oil vapor and then incubated for 48 hours at 37° C. No microbial colonies were observed in any of the plates exposed to oil vapor, suggesting that the antimicrobial substances work in a broad spectrum.

TABLE 17
Bacteria strains killed by oil vapor

| Strains tested | Classes |
|---|---|
| Mycobacterium smegmatis | Acid Fast |
| Mycobacterium phlei | Acid Fast |
| Salmonella typhimurium | Gram negative |
| Klebsiella pneumonia | Gram negative |
| Escherichia coli | Gram negative |
| Pseudomonas aeruginosa | Gram negative |
| Enterobacter cloacae | Gram negative |
| Shigella sonnei | Gram negative |
| Serratia marcescens | Gram negative |
| Bacillus subtilis | Gram positive |
| Bacillus stearothermophilus | Gram positive |
| Staphylococcus aureus | Gram positive |
| Staphylococcus epidermidis | Gram positive |
| Streptococcus mutans | Gram positive |

### EXAMPLE 8

### Quantification of Disinfectant Assay

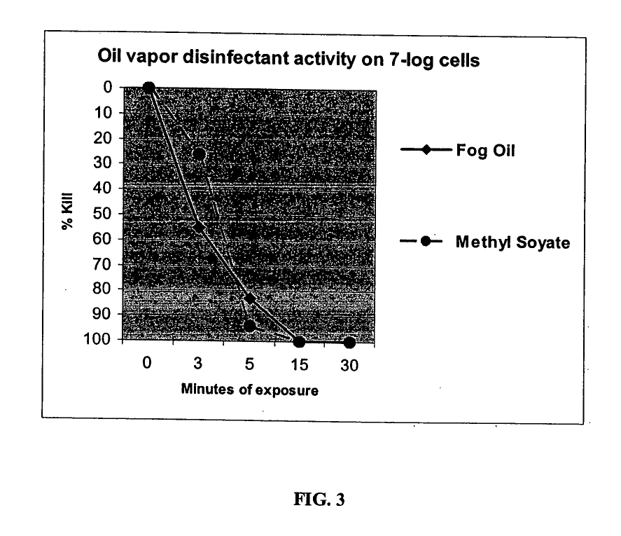

Quantification of the disinfectant activity was carried out using Salmonella. Approximately 10⁷ Salmonella cells were spotted onto the sterilized slide coverglass (size 1 inch×1 inch or 2.5 cm×2.5 cm). The inoculated coverglass was placed inside of petri dish and exposed to the vapor various times. After the exposure, the coverglass were transferred to the 50 ml conical tube containing 10 ml of T-soy broth and vortexed for 3 minutes to detach all the bacteria from the coverglass. Then serial dilutions were made to and 0.5 ml of each dilutions were plated onto the T-soy agar plate and incubated overnight at 37 C or until the colonies were visible. The total bacteria survived in the coverglass was calculated from the number of colonies counted on the plates where countable colonies were produced (which was less than 300 colonies). The percent kill rate with a given exposure time was calculated and is shown in FIG. 3.

### EXAMPLE 9

### Toxicity on Bacterial (Bacillus) and Fungal (Aspergillus niger) Spores

Three types of bacterial spore systems were exposed to disinfectant vapor. One system was the Duo-Spore™, which is commercially available from Propper Manufacturing Co, Inc, Long Island City, N.Y., and includes a mixture of Bacillus subtilis (3×10⁵) and Bacillus stearothermophilus (3×10⁶) spores on a paper strip. The spore strip was placed in the petri dish and exposed to the vapor with the lid open. another system was lab generated fungal spores:

The Duo-Spore™ strips were placed in nutrient broth allowing germination of Bacillus spores to vegetative cells. Vegetative cells were then incubated for 7 more days to induce the depletion of nutrients so that vegetative cells can develop into spores again. Nutrient broth containing newly formed spores were used for further testing after heating at 95° C. for 20 minutes to kill the residual vegetative cells. Aliquot of broth containing spores (total 10⁶ spores) was spotted onto Wattman paper, dried at 80° C. for 10 minutes, placed in the petri-dish, and exposed to the vapor with the lid open. 3). Bacillus anthracis-surrogates-Spore powder obtained from United States Army Chemical School at Fort Leonard Wood: The powder contained Bacillus spores, which Army uses as an alternative to the Bacillus anthracis (Anthrax) spores. 0.05 g of powder was placed in petri-dish and then incubated at 80° C. for 10 minutes before exposure to the vapor.

For the laboratory-generated fungal spores, Apergillus niger was inoculated into potato dextrose agar plates and allowed to sporulate for 2 weeks. After 2 weeks of incubation, the black spore clumps were transferred to a tube containing 10 ml of distilled water with 3 drops of kitchen detergent Ivory and vortexed for 3 minutes to disperse the spores. The number of the spores in the solution was also counted in the Petro-Hausser chamber and aliquot of spores (total 10⁶ spores) was spotted onto the potato dextrose agar plate and exposed to the vapor with the lid open.

Vapors were generated for 30 minutes, 60 minutes, and 90 minutes respectively, as in Example 1 for both bacterial and fungal spores,

For the Duo-Spore™ bacterial spores, the spore samples were transferred to the tube containing t-soy broth and incubated for up to 2 weeks at 37° C. The spore survivals were evaluated by the cloudiness of culture broth, which indicates the growth of bacterial cells after germination of spores.

Plates containing laboratory generated fungal spores were taken out after the exposure and incubated in the room for 2 weeks and observed for the visual growth of Aspergillus niger.

No bacterial and fungal growth was observed with the spore samples exposed more than 60 minutes suggesting that both mineral oil and methyl soyate vapors are sporicidal.

### EXAMPLE 10

### Carcinogenicity/Oxidative Stress Test on Rat

The carcinogenicity of the mineral oil and methyl soyate vapor-aerosols was examined on rats. Rats were exposed to air, mineral oil, and methyl soyate for 20 minutes and sacrificed 2 hours later, with subsequent analysis of lung tissues. Levels of DNA damage measured by the amount of 8-hydroxyguanine (8-oxodG), lipid peroxidation by-product called MDA (malondialdhyde), reduced form of Glutathione/Oxidized form of Glutathione (GSH/GSSG thiol level), and catalase activity, all of which are being used an indicators of carcinogenesis or oxidative stress, were examined and results are shown in Table 18.

TABLE 18
DNA Damage and Oxidative Stress Parameters In The Rat Lung

| | Air control | Mineral oil | Methyl soyate |
|---|---|---|---|
| 8-oxodG × 10⁵ | 1.52 ± 0.64 | 7.97 ± 0.39 | 0.98 ± 0.56 |
| GSH | 4.32 ± 0.16 | 4.30 ± 2.16 | 3.56 ± 1.02 |
| GSSG | 2.38 ± 0.16 | 1.37 + 0.64 | 0.87 + 0.09 |
| Catalase | 0.04 + 0.01 | 0.05 + 0.01 | 0.05 + 0.01 |
| MDA | 0.93 + 0.21 | 1.32 + 0.30 | 0.69 + 0.14 |

The study shows that oxidative stress indicators tested have not been increased with the exposure to either mineral oil and methyl soyate vapor-aerosols, however, mineral oil induced increases in DNA damage by 5 fold while methyl soyate did not.

1. A method of decontaminating an area for mitigation of a pathogen, wherein said method comprises the step of:
(a) dispersing an airborne biocidal oil to a contaminated area in an effective amount for decontamination purposes; and
(b) allowing the biocidal oil to mitigate the pathogen.

2. The method of claim 1, wherein the airborne biocidal oil comprises methyl soyate

3. The method of claim 2, wherein the pathogen is selected from the group consisting of virus, bacteria, fungus, and combinations thereof.

4. The method of claim 2, wherein the pathogen is a virus selected from the group consisting of influenza virus, West Nile virus, small pox virus, and combinations thereof.

5. The method of claim 2, wherein the pathogen is a species of bacteria selected from the group consisting Bacillus, Yersinia, Salmonella, Escherichia, Shigella, Pseudomona, Serratia, Enterobacter, Clostridium, Campylobacter, Klebsiella, Mycobacterium, Staphylococcus, Bordetella, Streptococcus, Francisella, Legionella, Vibrio and combinations thereof.

6. The method of claim 2, wherein the pathogen is a species of fungus selected from the group consisting of Blastomyces, Candida, Stachybotrys, Aspergillus, Acremonium, Histoplasma, Tinea, Fusarium, Ceratocystis, Cladisporium, Penicillium, and Botrytis.

7. The method of claim 2, wherein the step (a) of dispersing includes misting or vaporizing the methyl soyate.

8. The method of claim 2, wherein the step (a) of dispersing includes heating the methyl soyate to a temperature ranging from 350° C. to 650° C. and combining the methyl soyate with a compatible carrier gas.

9. The method of claim 2, wherein the effective amount of methyl soyate used in the dispersing step (a) includes at least about 0.05 mL of methyl soyate for each liter of inert carrier gas.

10. The method of claim 2, wherein the step (a) of dispersing includes misting the methyl soyate to provide aerosol particles having diameters ranging from 0.5 micron to 1.0 micron.

11. The method of claim 2, further comprising a step (c) of decontaminating vegetable matter that resides in the contaminated area.

12. The method of claim 2, further comprising a step (c) of decontaminating an animal or animal product that resides in the contaminated area.

13. The method of claim 2, wherein the dispersing step (a) includes dispersing the methyl soyate for a period of time ranging from 2 to 60 minutes.

14. The method of claim 2 further comprising a step (c) of mixing at least one additive with the methyl soyate.

15. The method of claim 14, wherein the additive is selected from the group consisting of coloring agents, carrier agents, scent agents, and combinations thereof.

16. A method of mitigating a pathogen, wherein said method comprises the steps of:
(a) adding liquid methyl soyate to a reservoir;
(b) pumping the liquid methyl soyate from said reservoir to a heated tubular heating element
(c) vaporizing said liquid methyl soyate in the tubular heating element;
(d) purging vaporized methyl soyate from said tubular furnace with an inert gas stream to provide a flowstream;
(e) condensing liquids from the flowstream; and
(f) dispersing the flowstream onto a contaminated object or into a contaminated area to mitigate the pathogen.

17. The method of claim 16, wherein said pathogen is selected from the group consisting of microbes, pathogens, parasites, and combinations thereof.

18. The method of claim 16, wherein the flowstream produced in the purging step (d) contains particles of the vaporized methyl soyate having diameters of about 0.5 micron to about 1.0 micron.

19. The method of claim 16, wherein the contaminated object in the dispersing step (f) is a plant.

20. The method of claim 16, wherein said contaminated object in the dispersing step (f) is an animal or animal product.

21. The method of claim 16, wherein the dispersing step (f) includes dispersing the methyl soyate for between about 2 and about 60 minutes.

22. The method of claim 16 further comprising a step of mixing at least one additive with the methyl soyate.

23. The method of claim 21, wherein the at least one additive is selected from the group consisting of coloring agents, carrier agents, scent agents, and combinations thereof.

24. A decontamination system comprising:
a smoke generator provided with methyl soyate material for use in generating smoke,
the smoke generator being configured for dispersion of the methyl soyate material to a contaminated area in an effective amount for decontamination purposes.

25. The decontamination system of claim 24, wherein the methyl soyate material includes methyl soyate and at least one additive.

26. The misting device of claim 25, wherein the at least one additive is selected from the group consisting of coloring agents, carrier agents, scent agents, and combinations thereof.
